(19) United States
(12) Patent Application Publication
(10) Pub. No.: US 2025/0241679 A1
(43) Pub. Date: Jul. 31, 2025

(54) INSERTION SYSTEM AND METHOD FOR INSERTING A MEDICAL DEVICE

(71) Applicant: Roche Diabetes Care, Inc., Indianapolis, IN (US)

(72) Inventor: Olaf Roscher, Mannheim (DE)

(21) Appl. No.: 19/183,038

(22) Filed: Apr. 18, 2025

Related U.S. Application Data
(63) Continuation of application No. PCT/EP2023/079091, filed on Oct. 19, 2023.

(30) Foreign Application Priority Data
Oct. 21, 2022 (EP) 22 202 923.3

(51) Int. Cl. A61B 17/34; A61B 5/145

(52) U.S. Cl. CPC A61B 17/3468; A61B 5/14503; A61B 2560/063

(57) ABSTRACT

An insertion device for inserting a medical device into a subject is disclosed. The insertion device has a chamber for receiving the medical device, an insertion tool for inserting an insertable portion of the medical device, an insertion mechanism, a retraction mechanism for retracting the insertion tool from the subject after insertion, a retaining mechanism for retaining the insertion tool in an end position after retraction, an inner housing having the retaining mechanism and the retracted insertion tool in the end position, an inserter housing having the insertion mechanism as well as a chamber for receiving the inner housing. The inserter housing is formed of recyclable or biodegradable or compostable material, the insertion device has an ejection mechanism to remove the inner housing from the chamber when the insertion tool is retrained within the inner housing by the retaining mechanism, and the ejection mechanism is activatable by the user.

### RELATED APPLICATIONS

This application is a continuation of International Application No. PCT/EP2023/079091, filed Oct. 19, 2023, which claims priority to European Application No. 22 202 923.3, filed Oct. 21, 2022, the entire disclosures of both of which are hereby incorporated herein by reference.

### BACKGROUND

The present application refers to an insertion device for inserting at least a part of a medical device into a subject comprising a chamber for receiving the medical device, an insertion tool for inserting the medical device or an insertable portion of the medical device, an insertion mechanism for penetrating the insertion tool into a subject and therewith inserting at the medical device or the insertable portion of the medical device into the subject, a retraction mechanism for retracting the insertion tool from the subject after insertion, a retaining mechanism for retaining the insertion tool in an end position after retraction, an inner housing comprising the retaining mechanism and the retracted insertion tool in the end position, and an inserter housing comprising the insertion mechanism as well as a chamber for receiving the inner housing.

For the proper management of chronic health conditions, it may be crucial for a subject to periodically monitor one or more analyte levels in his or her blood stream. In the case of diabetes the subject routinely monitors the glucose levels to avoid hypoglycemic and hyperglycemic episodes. For other situations where health monitoring is important, other analytes, such as lactate or oxygen, may be measured.

Systems have been developed which allow a sensor to be implanted into a subject to monitor the analyte concentration directly within the bloodstream or within the interstitial fluids. For instance, an electrochemical sensor may be inserted into a subcutaneous region of the subject where the analyte concentration is continuously monitored and/or logged.

For instance, U.S. Pat. No. 10,653,445 B2 discloses a sensor insertion assembly for inserting a sensor with an inserter having a chamber for removably receiving a sensor cartridge, wherein the sensor cartridge comprises an insertion needle with a sensor inside, the needle disposed within a sealed sterile capsule. The inserter further comprises an actuator for actuating the insertion. After the insertion, the cartridge may be removed and the inserter reused with a new cartridge unit. The used cartridge shields the needle after removal preventing anyone removing and handling the cartridge to come into contact with the used needle. Another insertion assembly with a reusable inserter is described in U.S. Pat. No. 9,883,888 B2 disclosing a sensor cartridge with a sterile capsule comprising a needle as well as a retracting mechanism.

U.S. Pat. No. 10,945,649 B2 discloses a reusable insertion assembly, wherein the insertion assembly is configured to receive a needle hub and a sensor hub, which are transferred to the inserter with the help of a carrier. The carrier may also be used to remove the needle hub from the inserter after insertion of the sensor, but the carrier is neither keeping the needle in a sterile environment before transferring the needle to the inserter nor sealing it when used for removal of the needle.

A further insertion assembly with a reusable insertion gun is known from U.S. Pat. No. 6,579,690 B1.

U.S. Pat. No. 10,737,021 B2 discloses inserter for inserting a part of a medical system, such as a cannula of an insulin pump, wherein the needle is capsuled in an inner housing before it is inserted into the inserter.

The reusable design of an insertion device does have the advantage of a reduced amount of waste per application. This advantage comes at the cost of a higher effort regarding production as well as during use as the user has first to prepare the inserter assembly before using.

U.S. Publication No. 2021/0052302 A1 discloses a disposable biosensor inserter assembly with a carrier for the bio sensor, an insertion device and a retraction mechanism positioned inside a housing consisting. After insertion the retraction mechanism prevents reinsertion of the insertion device fixedly positioning the insertion device within the housing. The housing may be formed of recyclable or biodegradable or compostable material.

The advantage of the completely disposable design is the lower effort and more convenient handling for the user but the garbage produced and the effort of disposing of the inserter device are typically higher.

### SUMMARY

The disadvantages of the prior art are addressed by providing a mechanism to separate recyclable or biodegradable parts of an insertion device from encapsulated sharp and/or contaminated parts after use of the insertion device. Advantageous embodiments which might be realized in an isolated fashion or in any arbitrary combinations are listed in the list of embodiments and throughout the specification.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may all refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “housing,” “opening,” and “chamber,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of this disclosure, an insertion device for inserting at least a part of a medical device into a subject is disclosed. The insertion device comprises a chamber for receiving the medical device, an insertion tool for inserting the medical device or an insertable portion of the medical device, an insertion mechanism for penetrating the insertion tool into the subject and therewith inserting the medical device or the insertable portion of the medical device into the subject, a retraction mechanism for retracting the insertion tool from the subject into an end position after insertion, a retaining mechanism for retaining the insertion tool in the end position after retraction, an inner housing comprising the retaining mechanism and the retracted insertion tool in the end position, and an inserter housing comprising the insertion mechanism as well as the inner housing. The inserter housing is formed of recyclable or biodegradable or compostable material. Furthermore, the insertion device comprises an ejection mechanism to remove the inner housing from the inserter housing when the insertion tool is retrained within the inner housing by the retaining mechanism, and the ejection mechanism is activatable by the user.

The term “insertion device” refers to a device configured for inserting the medical device fully or partially into the body tissue. The insertion device may be configured for transcutaneously or subcutaneously inserting the medical device or a part of the medical device into the body tissue, such as by performing an incision or a puncture in a skin of the user and by transferring the medical device fully or partially into the body tissue.

The term “inserting” refers to an action or process of one or more of transcutaneously or subcutaneously implanting and/or positioning the medical device into the body tissue of the user. The medical device, such as the analyte sensor or a drug delivery device, may fully or partially be inserted into the body tissue. The insertion of the medical device may be performed by using the insertion device. After insertion, the medical device or at least a part of the medical device may remain in the body tissue of the user for a predetermined period of time, such as for several hours, or days or weeks.

The term “medical device” refers to an arbitrary element or article being configured for use in the field of medical technology, specifically in the field of medical analytics or medical diagnostics. The medical device may be configured for performing at least one medical function and/or for being used in at least one medical process, such as one or more of a therapeutic process, a diagnostic process or another medical process.

For example, the medical device may comprise at least one of: a device for delivery of at least one therapeutic fluid, for example, insulin, into the body tissue of the user, specifically at least one infusion set comprising at least one infusion cannula; at least one analyte sensor for detecting at least one analyte, for example, blood glucose or a pH value; specifically at least one analyte sensor for detecting at least one bodily analyte in the at least one bodily fluid, more specifically at least one electrochemical analyte sensor comprising at least one sensor electrode for detecting the at least one analyte.

“Chamber” for receiving the medical device refers to a void or cavity within the insertion device in which the medical device may be positioned before an insertion step. The chamber may, for example, be a void within the inner housing or a void within the inserter housing next to the inner housing. In some embodiments the chamber comprises holding means holding or fixing the medical device within the chamber before the insertion, and releasing the medical device during or after the insertion step.

“Insertion tool” refers to a sharp or at least solid member suitable to enter the skin for inserting a medical device, such as needle or cannula with a hollow tube and a sharp tip. The insertion tool is considered as part of the insertion device. However, in particular during manufacturing of the insertion device, the insertion tool may also be considered as a part of the medical device. For example, the insertable part of the medical device such as a sensor and/or sensor electrodes may be positioned within the insertion tool, e.g., a needle with a longitudinal slit.

“Insertion mechanism” refers to a mechanism and/or components for inserting the medical device fully or partially into the body tissue. In other words the insertion mechanism is arranged to move the medical device from a first position, a proximal position, to a second position, a distal position, wherein in the first position the medical device is completely located within the insertion device, e.g., at a proximal end of the insertion device, and in the second position at least a part of the medical device is located outside of the insertion device, e.g., protruding from a distal opening of the insertion device. The movement may be caused by a force exerted by the user and/or a spring or the like.

In some embodiments the medical device comprises an on-body-unit and a sensor protruding from a lower surface of the on-body-unit wherein in the second position the on-body-unit is positioned at a distal end of the insertion device so that the lower surface of the on-body-unit is located within the distal opening of the insertion device and the sensor protrudes the insertion devices.

The inserter housing may be comprised of one or two or more parts. For example, the inserter housing comprises an insertion cap and a contact sleeve wherein the insertion cap may be part of the insertion mechanism. For example, the insertion cap is movable relative to the contact sleeve moving or pushing the medical device from the proximal position within the contact sleeve to a distal position wherein the medical device at least partially protrudes a distal opening of the contact sleeve.

“Retraction mechanism” (also referred to herein as “retractor”) refers to a mechanism and/or one or more components for retracting the insertion tool from the body tissue of the user after the insertion of the medical device, i.e., a mechanism moving the insertion tool from the distal position at least partially protruding the insertion device to a proximal position inside the insertion device, the end position. The insertion tool may be retracted deeply into the needle housing of the insertion device to minimize the infection risk. The retraction of the insertion tool may be initiated by the user or automatically after an insertion movement of the insertion tool into the skin.

“End position” refers to a position the insertion tool reaches after retraction, in which the insertion tool is fully comprised within the inner housing.

“Retaining mechanism” (also referred to herein as “retainer”) refers to a mechanism and/or one or more components for retaining the insertion tool within the insertion device after the insertion tool has been retracted by the retraction mechanism. In some embodiments the retaining mechanism comprises locking means to fix the insertion tool in the position reached after the insertion tool has been retracted by the retraction mechanism.

The “inner housing” refers to a housing that at least comprises the retaining mechanism as well as the insertion tool in the end position reached after retraction and that prevents any further contact with the insertion tool. The inner housing is encapsulating the insertion tool thus the sharp and contaminated parts guaranteeing a safe handling of the insertion tool when disassembling the insertion device.

“Recyclable or biodegradable or compostable material” refers to any materials that after use may be converted into new material, or may be biodegraded, thus broken down by microorganisms, such as bacteria and fungi, or may be composted.

“Ejection mechanism” (also referred to herein as “ejector”) refers to a mechanism, i.e., one or more components, configured to disassemble the insertion device, and in particular to separate the inner housing from the rest of the inserter medical device, in particular from the inserter housing. That way the insertion tool may be safely disposed and the rest of the insertion device, in particular, the inserter housing, may be recycled or composted thereby significantly reducing the environmental burden of the overall system.

“Activatable” refers to being configured to activate and/or trigger a function, e.g., to trigger a mechanism such as the ejection mechanism to operate. With the ejection mechanism being activatable by the user, the user may determine the point in time the insertion device is disassembled.

An advantage of the inventive insertion device is the improved sustainability due to using recyclable material where possible. A further advantage is the safety and easy handling that is maintained due to the inventive concept of encapsulating any sharp and/or contaminated parts and providing a mechanism to easily and safely disassemble the different portions of the inventive insertion device. No extra tools are necessary and there is no risk of contacting the sharp.

The insertion device may also include where the ejection mechanism is configured to withdraw at least one fastener from a stop position.

“Fastener” refers to a device and/or a means for fixing a component in a defined position. A fastener such as a pin may be an easy way of securing the inner housing in a fixed position within the inserter housing during use. The ejection mechanism may then comprise at least one tool, part and/or mechanism to move the fastener from the first position, also referred to as stop position, securing the inner housing to a second position, wherein the inner housing may freely move relative to the inserter housing when the fastener is in the second position. The inner housing may then be unhinched from the inserter housing, e.g., by shear force.

The insertion device may also include where for activating the ejection mechanism the inserter housing includes at least one push button.

In another embodiment the ejection mechanism further comprises a securing device that has to be removed and/or activated to be able to push the push button to prevent unintentional activation of the ejection mechanism. In an alternative embodiment the ejection mechanism comprises two push buttons that have to be pushed simultaneously to activate the ejection mechanism. Other security measures may be comprised by the ejection mechanism to prevent unintentional activation of the ejection mechanism and/or to prevent an activation of the ejection mechanism before an insertion process has occurred, i.e., before the insertion tool has been inserted, retracted and retained in the end position by the corresponding mechanisms.

The insertion device may also include at least one opening to at least partially insert an activation tool into the inserter housing for activating the ejection mechanism. “Activation tool” refers to any part or component configured to trigger the ejection mechanism. For example, the activation tool may comprise a stick-like part that may be inserted through the opening to activate the ejection mechanism.

Therewith, the ejection mechanism may be fully retrieved within the housing, reducing or suspending the risk of inadvertent actuation.

The insertion device may also include where the insertion device includes a removable component forming the activation tool when removed from the insertion device.

For example, a part of the inserter housing may comprise an elongated part, such as a stick or finger or the like, which may be easily removed from the insertion device, i.e., form the removable component, and function as activation tool.

The insertion device may also include where the removable component of the insertion device is a sealing cap or part of a sealing cap.

“Sealing cap” or safety cap refers to a cap that covers a distal opening of the insertion device as long as the insertion device is not used. The sealing cap may comprise an elongated part protruding from the sealing cap after it is removed from the insertion device, e.g., by pulling or rotating the cap relative to the rest of the insertion device.

The insertion device may also include where the at least one opening of the inserter housing is sealed by a removable or breakable cover.

For example, the opening may be sealed with an adhesive label which may be removed or pierced by the activation tool. The cover may help to improve the hygiene preventing any contamination to enter via the opening.

The insertion device may also include where the chamber for receiving the medical device is comprised by the inner housing.

Another alternative is to position the medical device within a chamber within the inserter housing that is not comprised by the inner housing.

In one aspect, a kit includes an insertion device of any of the preceding claims and a medical device.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 schematically illustrates an insertion device in accordance with a first embodiment;

FIG. 2 schematically illustrates an insertion device in accordance with a second embodiment;

FIG. 3 illustrates a sectional view of an insertion device in accordance with a further embodiment;

FIG. 4 illustrates an activation tool in accordance with one embodiment;

FIG. 5 illustrates an outer appearance of an insertion device before use in accordance with one embodiment;

FIG. 6 illustrates a sectional view of an insertion device during an insertion process in accordance with one embodiment;

FIG. 7 illustrates a sectional view of an insertion device after an insertion process in accordance with one embodiment; and

FIG. 8 illustrates a sectional view of an inner housing after an ejection process in accordance with one embodiment.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

To easily identify the discussion of any particular element or act, the most significant digit or digits in a reference number refer to the figure number in which that element is first introduced.

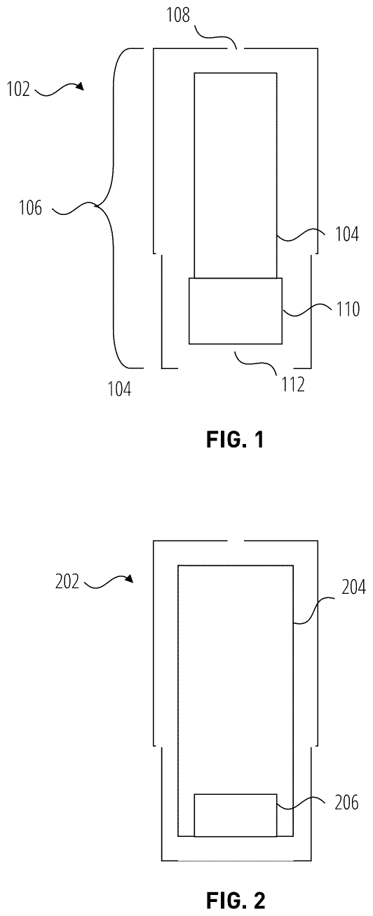

FIG. 1 is a schematic view of certain aspects of an insertion device 102 for inserting at least a part of a medical device into a subject according to a first embodiment. The insertion device 102 comprises an inner housing 104 encapsulating at least an insertion tool (not shown) and a retaining mechanism to retain the insertion tool within the inner housing 104 after an insertion process. For the insertion process the inner housing 104 is positioned within an inserter housing 106 that consists of recyclable or biodegradable or compostable material. Via an ejection mechanism, e.g., inserting an activation tool (not shown) through an opening 108 on a distal end of the inserter housing 106, the inner housing 104 is separable from the inserter housing 106, so that the sharp and/or contaminated parts can be safely disposed separate from the recyclable or biodegradable parts. The ejection mechanism is configured to be activated by a user. The insertion device 102 further comprises a chamber 110 within the inserter housing 106 between a proximal opening 112 and the inner housing 104 to receive the medical device.

Alternatively, as shown in FIG. 2, the chamber 206 is comprised by the inner housing 204 of the insertion device 202, so that the medical device may be received by the inner housing 204 before the insertion process.

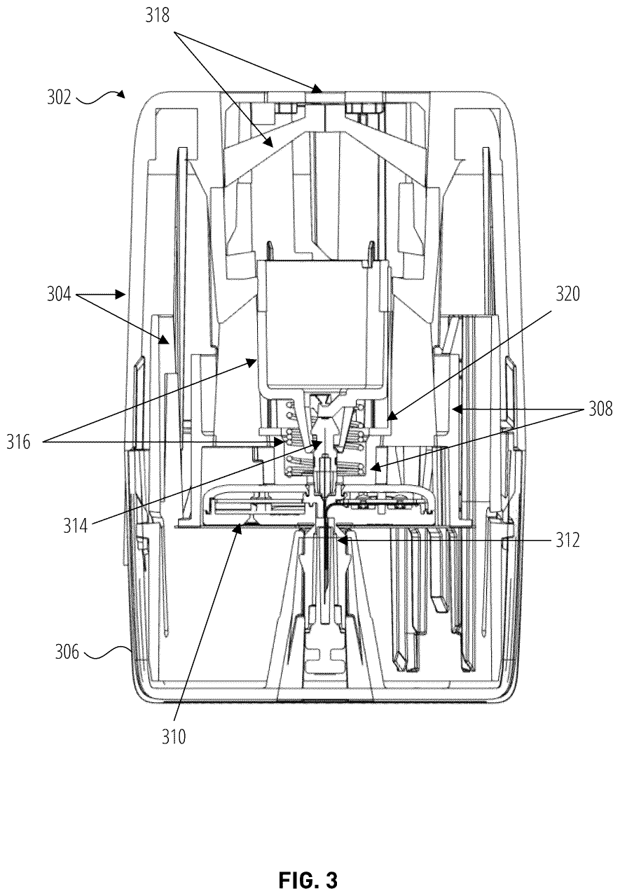

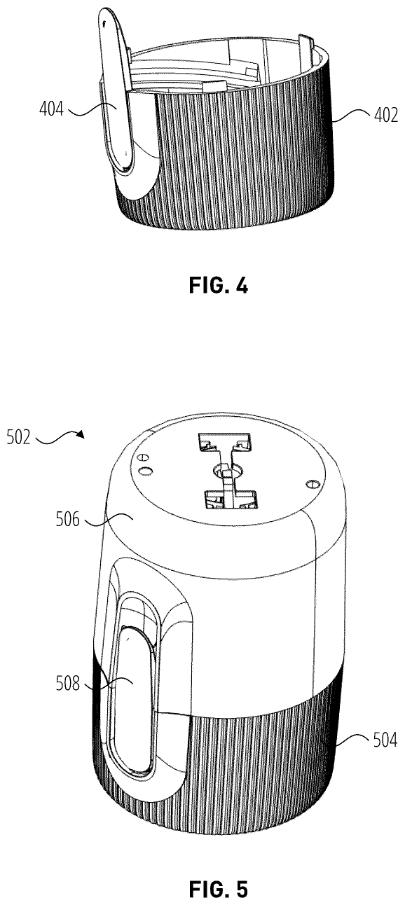

FIG. 3 shows a sectional view of aspects of kit comprising an insertion device 302 and a medical device 310 according to an embodiment. The insertion device 302 is depicted before use, wherein an inserter housing 304 is combined with a sealing cap 306 to close the proximal opening of the inserter housing 304. Preferably, the sealing cap 306 is also made of a recyclable or biodegradable or compostable material. An inner housing 308 is positioned within the inserter housing 304 and a medical device 310 is positioned within a proximal opening of the inner housing 308, wherein an insertable part of the medical device 310, e.g., sensor electrodes, are positioned within an insertion tool 312, such as a longitudinally slit needle, that protrudes through an opening of the medical device 310 and into the sealing cap 306. A distal end of the insertion tool 312 is fixed in a needle hub 314 within the inner housing 308.

A retraction mechanism 316 to retract the insertion tool 312 after insertion is realized by a retractor holding the needle hub 314 and a pre-stressed retraction spring. Together with a locking structure 320 to lock the retractor in a retracted position the retraction mechanism 316 also forms part of the retaining mechanism retaining the insertion tool in an end position within the inner housing 308 after retraction.

An ejection mechanism 318 is realized by an opening in a proximal wall of the inserter housing 304 and two flexible arms comprised by the inserter housing 304 that can be reached through the opening, e.g., by an activation tool (not shown) and releasing the inner housing 308 from the inserter housing 304 when pressed down.

FIG. 4 shows an embodiment of a sealing cap 402 as a removable component having an elongated arm protruding from a side wall. The arm can function as an activation tool 404 that can be inserted through proximal wall opening activating the ejection mechanism 318 by pressing the flexible arms down.

FIG. 5 shows an outer appearance of the insertion device 502 with the sealing cap 504 attached to the inserter housing 506, so that the protruding arm like activation tool 508 reaches over a part of the inserter housing 506.

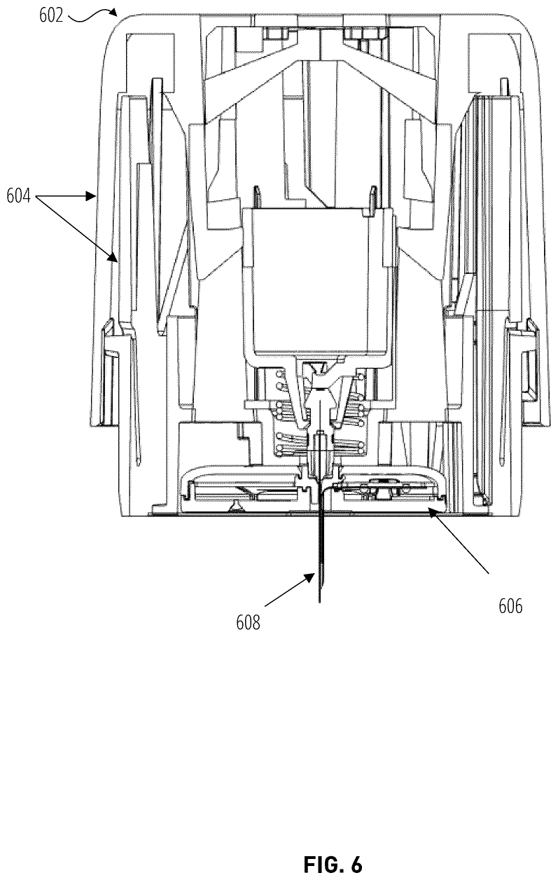

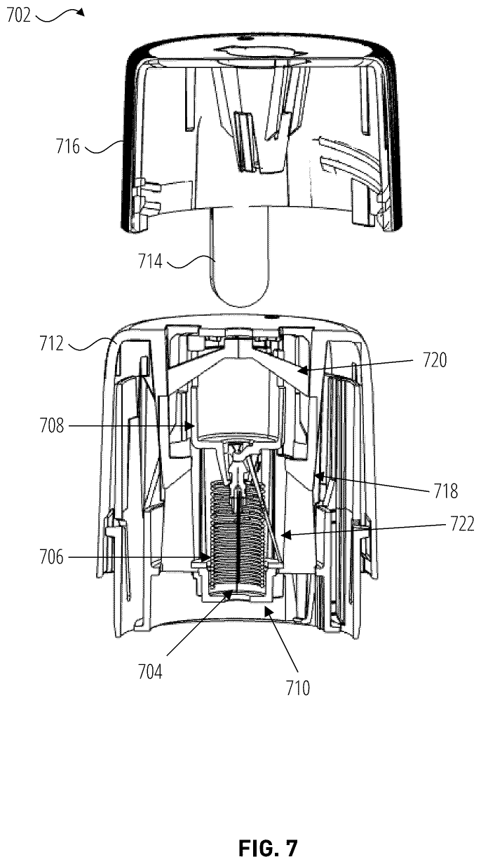

FIG. 6 shows the insertion device 602 during the insertion process. The sealing cap is removed, the inner part of the inserter housing 604 is positioned on an insertion site. The outer cap part of the inserter housing 604 is pushed down in a proximal direction therewith moving the medical device 606 and the insertion tool 608 down so that the insertion tool 608 is protruding from the inserter housing 604 and inserted into a subject at the insertion site.

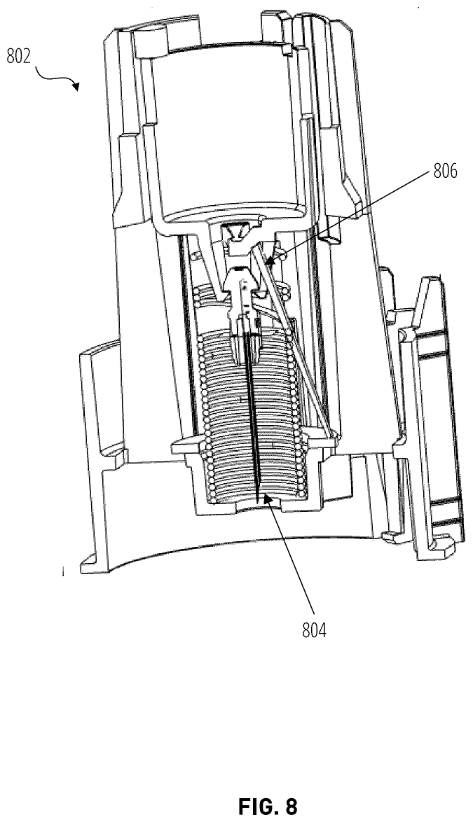

FIG. 7 shows the insertion device 702 after the insertion process. The insertion tool 704 is retracted from the insertion site by the retraction mechanism, namely by the retraction spring 706 moving the retractor 708 in a distal direction, and retained by the retaining mechanism 722, namely the retractor 708 being locked in the retracted position to fix or retain the insertion tool 704 in the end position. The inner housing 710 may now be released from the inserter housing 712 by inserting the arm-like activation tool 714 of the sealing cap 716 through the opening to push two lever arms 720 of the ejection mechanism down therewith loosening lateral retaining hooks 718 to release the inner housing 710 from the inserter housing 712. The lateral retaining hooks 718 function as a fastener fixing the inner housing 710 within the inserter housing 712 when in a first position, the so called stop position, and releasing the inner housing 710 when withdrawn from the stop position into a second laterally expanded position after activation of the ejection mechanism.

FIG. 8 shows the inner housing 802 after being released from the inserter housing by the ejection mechanism. The ejected inner housing 802 comprises the insertion tool 804 retained within the inner housing 802 by the retaining mechanism 806.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LISTING OF DRAWING ELEMENTS

102 insertion device
104 inner housing
106 inserter housing
108 opening
110 chamber
112 proximal opening
202 insertion device
204 inner housing
206 chamber
302 insertion device
304 inserter housing
306 sealing cap
308 inner housing
310 medical device
312 insertion tool
314 needle hub
316 retraction mechanism
318 ejection mechanism
320 locking structure
402 sealing cap
404 activation tool
502 insertion device
504 sealing cap
506 inserter housing
508 activation tool
602 insertion device
604 inserter housing
606 medical device
608 insertion tool
702 insertion device
704 insertion tool
706 retraction spring
708 retractor
710 inner housing
712 inserter housing
714 activation tool
716 sealing cap
718 lateral retaining hooks
720 lever arm
722 retaining mechanism
802 inner housing
804 insertion tool
806 retaining mechanism

1. An insertion device for inserting at least a part of a medical device into a subject, comprising:
a chamber configured for receiving the medical device;
an insertion tool configured for inserting the medical device or an insertable portion of the medical device;
an insertion mechanism configured for penetrating the insertion tool into the subject and therewith inserting the medical device or the insertable portion of the medical device into the subject;
a retraction mechanism configured for retracting the insertion tool from the subject into an end position after insertion;
a retaining mechanism configured for retaining the insertion tool in the end position after retraction;
an inner housing comprising the retaining mechanism and the retracted insertion tool in the end position; and
an inserter housing comprising the insertion mechanism as well as the inner housing;
wherein:
the inserter housing is formed of recyclable or biodegradable or compostable material,
the insertion device comprises an ejection mechanism to remove the inner housing from the inserter housing when the insertion tool is retained within the inner housing by the retaining mechanism, and
the ejection mechanism is activatable by the user.

2. The insertion device of claim 1, wherein the ejection mechanism is configured to withdraw at least one fastener from a stop position.

3. The insertion device of claim 2, wherein the inserter housing comprises at least one push button configured for activating the ejection mechanism.

4. The insertion device of claim 2, further comprising at least one opening to at least partially insert an activation tool into the inserter housing for activating the ejection mechanism.

5. The insertion device of claim 4, wherein the insertion device comprises a removable component forming the activation tool when removed from the insertion device.

6. The insertion device of claim 5, wherein the removable component of the insertion device is a sealing cap or part of a sealing cap.

7. The insertion device of claim 4, wherein the at least one opening of the inserter housing is sealed by a removable or breakable cover.

8. The insertion device of claim 1, wherein the chamber for receiving the medical device is comprised by the inner housing.

9. A kit comprising the insertion device of claim 1 and a medical device.
